(19) United States
(12) Patent Application Publication
(10) Pub. No.: US 2022/0253140 A1
(43) Pub. Date: Aug. 11, 2022

(54) MYOELECTRIC WEARABLE SYSTEM FOR FINGER MOVEMENT RECOGNITION

(71) Applicant: BRINK BIONICS INC., Waterloo (CA)

(72) Inventors: Erik LLOYD, Waterloo (CA); Ning JIANG, Waterloo (CA); Jiayuan HE, Waterloo (CA); Auguste KOH, Waterloo (CA); Tushar CHOPRA, Waterloo (CA)

(21) Appl. No.: 17/624,542

(22) PCT Filed: Jul. 3, 2020
(86) PCT No.: PCT/CA2020/050935

§ 371 Date: Jan. 3, 2022

Related U.S. Application Data
(60) Provisional application No. 62/870,626, filed on Jul. 3, 2019.

(51) Int. Cl. G06F 3/01; G06F 3/038; G06F 1/16; G06N 20/00

(52) U.S. Cl. CPC G06F 3/015; G06F 3/017; G06N 20/00; G06F 3/0383; G06F 1/163; G06F 3/014

(57) ABSTRACT

A user-wearable system for communicating with a computer system includes a hand wearable device, a sensor array on the hand wearable device and configured to capture myoelectric signals from the user's hand and a means for converting the myoelectric signals into computer-readable signals corresponding to a computer instruction executed by the computer system.

### FIELD OF THE INVENTION

The disclosure relates to the field of human-machine interaction, and specifically to a myoelectric wearable 102 system for finger movement recognition.

### BACKGROUND OF THE INVENTION

Electromyogram (EMG) or myoelectric signals are the electrical manifestations of skeletal muscle activities. Normally, the signals can be measured noninvasively by attaching the electrodes (sensors) on the skin above the muscles. As the signals containing neural information of muscle contractions, they can be processed, such as by machine learning algorithms, to infer the motion intentions of the user, and then mapped to the command of the machines or the electronics, such as advanced prosthetic hands, robotics, augmented reality (AR) and virtual reality (VR) equipment, personal computers (PCs) and drones, to achieve gesture control.

The control of the advanced prosthetic hands is the traditional application of myoelectric control. Normally the electrodes are attached on the residual of the forearm. The amputee can contract the residual muscles, where the signals are collected and analyzed to control the activation of the powered prosthetic hand, functioning as a replacement of the lost hand. Recently, commercial myoelectric armbands, such as products from Thalmic Labs (now North) and CTRL-Labs (now acquired by Facebook), have been developed for normal people as a general tool to achieve the control of machines or electronics with myoelectric signals. The armbands usually contain eight or more electrodes and are worn on the forearm of the user. To increase the accuracy of the hand movement estimation, except for the myoelectric electrodes, inertial measurement unit (IMU) sensors are usually embedded in the system as well.

In the above-mentioned devices, they both attach the electrodes on the forearm, i.e. the area between the wrist and the elbow. The collected signals are mainly used to estimate the movements of the fingers. However, the surface EMG (sEMG) signals, which are collected by the electrodes on the skin, are a mix of activities from multiple muscles. As the muscles relevant to finger movements, such as flexor digitorum superficialis muscle (FDS) and flexor digitorum profundus muscle (FDP), either small or deeply buried, it is hard to separate their signals from others and extract the information to infer the wanted finger movements. As such, it is hard to accurately estimate finger movements from the electrodes attached on the forearm. Further, as there are no positioning settings for the electrodes, it is hard to position the electrodes in the same place in each donning, which makes it difficult to reuse the pre-collected data in algorithms, resulting in a new round of data collection.

### SUMMARY OF THE INVENTION

In one embodiment of the invention, there is disclosed a user-wearable system for communicating with a computer system comprising a hand wearable device; a sensor array on the hand wearable device and configured to capture myoelectric signals and kinematic data from the user's hand; and a means for converting the signals from the sensor array into computer-readable signals corresponding to a computer instruction executed by the computer system. The sensor array is positioned on the hand wearable device such that it is on a dorsal side of the user's hand when worn

In one aspect of the invention, the means for converting comprises a control module on the hand wearable device.

In another aspect of the invention, the control module is configured to manage the data acquisition of each sensor on the sensor array and encode data received from each sensor.

In another aspect of the invention, there is a transmission module for sending data to the computer system.

In another aspect of the invention, the transmission module communicates with the computer system by a wired, such as USB, or a wireless, such as BLE, WIFI, communication protocol.

In another aspect of the invention, the means for converting comprises machine learning algorithms, such as artificial neural networks (ANN), executed by the computer processor to decode data received from each sensor into the movements of the hand wearable.

In another aspect of the invention, the machine learning algorithms is calibrated in response to an initial capture of data by executing a calibration tool which provides the user with target movements of the hand wearable to correspond with visual cues on a graphical user interface, or the data stored in the graphical user interface.

In another aspect of the invention, the means for converting includes a map between the movements of the hand wearable and the computer instructions which is configured in the graphical user interface by the user.

In another aspect of the invention, the graphical user interface translates the estimated movements from the machine learning algorithm to the computer-readable signals which corresponds with computer instructions based on the configured mapping.

In another aspect of the invention, the computer instructions include mouse click and/or key/button press equivalent functions.

In another aspect of the invention, there is a sensor array for capturing myoelectric signals from the back of the hand, and the contacts for sensing is made of hard metals, such as silver or gold, or soft conductive materials, such as conductive silicone.

In another aspect of the invention, there is a second sensor array for capturing kinematic data.

In another aspect of the invention, the myoelectric signal and the kinematic data are converted to digital signals with a sampling rate above 1000 Hz.

In another aspect of the invention, the hand wearable device consists of a textile with a first loop for an index finger and a second loop for a thumb.

In another aspect of the invention, the sensor array is positioned with respect to the finger loops.

In another aspect of the invention, the hand wearable device further includes a first adjustment strap at a side of the user's hand and a second adjustment strap around the base of the user's thumb.

### BRIEF DESCRIPTION OF THE DRAWINGS

Embodiments will now be described, by way of example only, with reference to the attached Figures, wherein:

FIGS. 1A and 1B show front and rear views of a hand-wearable device according to embodiments of the invention.

FIG. 2 is a schematic view of a system according to embodiments of the invention.

### DETAILED DESCRIPTION

The applicant discloses a myoelectric hand wearable system 108, 110, 112 that may be worn on the hand of a user. The system includes a device designed as a tool to recognize finger movement via surface electromyogram (sEMG) signals and kinematic data. The system could help users achieve control of machines and electronics, which are connected with the proposed system, by mapping the estimated finger movements to specific commands of the connected system. Different from prior art armbands, the sEMG electrodes are attached on the back of the hand, instead of the forearm, and the positioning settings are designed accordingly. As there are many muscles related to the finger movement on the back of the hand, and they are all superficial muscles, the information extracted from these sEMG signals would be beneficial for the accurate estimation of finger movements. In addition, the thumb and index finger are designed as anchors to position the electrodes, which reduces the difference among each donning and doffing and helps to reuse pre-collected data in algorithms.

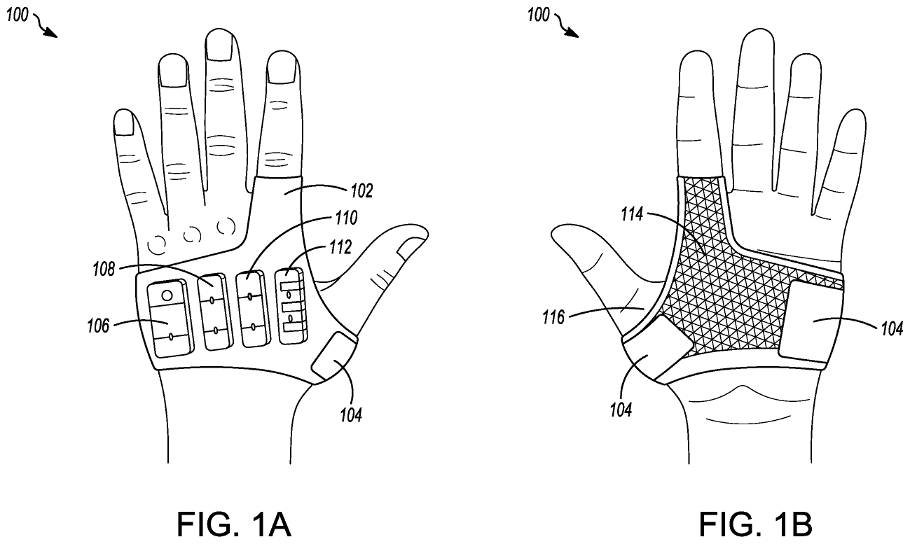

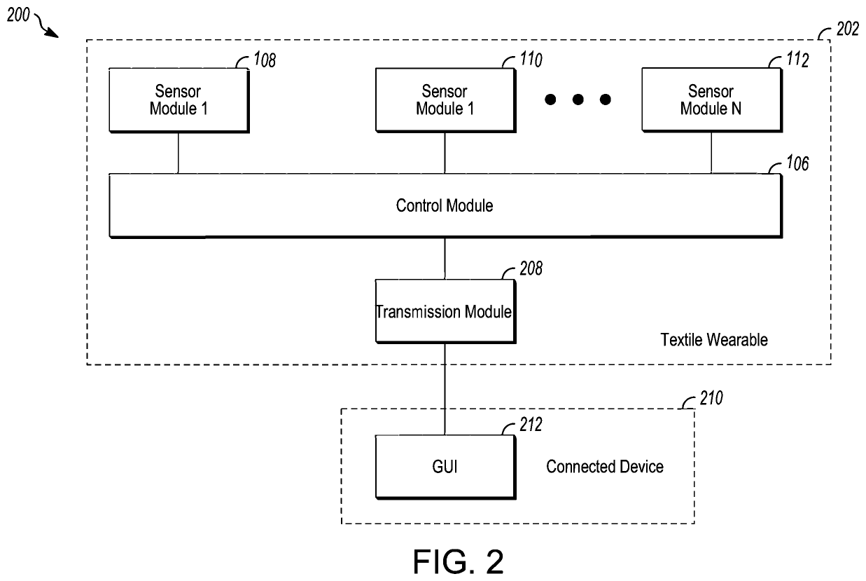

Referring now the drawings, an example hand wearable device and associated components are shown in FIGS. 1A and 1B, and a schematic block diagram is shown in FIG. 2. The hand wearable device is preferably a textile hand wearable device. The system includes, a sensor array or module 108, 100, 112, a control module 106, a transmission module 208 (or as part of control module 106), a graphical user interface (GUI) 212 and the textile wearable 108, 110, 112. The sensor module 108, 110, 112, control module 106 and transmission module 40 are all housed in or on the textile wearable 102. The GUI 212 is installed in software in connected machines or electronics. The sensor array position on the hand wearable device such that it is on a dorsal side of the user's hand when worn.

The sensor module 108, 110, 112 is used to capture electrical signals from the skin, amplify and filter the raw data to get high-quality signals and digitize the data for further processing. The contacts in each sensor module 108, 110, 112 to sense electrical activities are be made of hard metals, such as silver or gold, or soft conductive materials, such as conductive silicone. The signals captured by the contacts are differentially amplified in each sensor module 108, 110, 112 to reduce the interference, restrain the common model signal, and enlarge the differential mode signal. The differential signal is high pass filtered. The cutoff frequency of the filter is between 10 and 20 Hz. The IMU-based sensors are also embedded in module 108, 110, 112 to capture the kinematic information, such as velocity, acceleration. The sEMG signal and kinematic data is converted to the digital signal with a sampling rate above 1000 Hz. The digital signal is then transmitted to the controller module for further processing. The sensor module '108, 110, 112 is designed to run independently. As such, the damage of one sensor would not affect the running of the others. The number of sensor modules is flexible based on the demands of the use.

The controller module 106 manages the data acquisition of each sensor module 108, 110, 112, process and encodes data received from the sensor module 108, 110, 112, and controls the transmission module 208 to send the data to the connected machine or electronics. The data processing task implemented here can be entire or part of the algorithm for finger movement recognition depending on the complexity of the algorithm. If the algorithm is complicated and out of the computational capacity of the controller, the data processing will be implemented in the GUI 212 installed in the connected device.

The transmission module 208 sends the encoded data to the connected machine. Currently a UART (universal asynchronous transmitter receiver) serial communication is adopted to send the data via a USB port to the PC, which also powers the system for they are wired. Wireless communication, such as BLE, WIFI, would also apply to the system. A power module (not shown) is needed if the wireless method is employed.

The above three modules are all housed in the textile wearable. The system is designed as a glove with adjustable straps. The index and thumb loops 102, 104 are purposed as the anchors to ensure the sensor modules 108, 110, 112 are correctly located in the intended places of the user's hand every time they put the glove on. The adjustable straps 104 are used to keep the sensor-skin contact firmly with different sizes of hands. One adjustable strap 104 is located at the side of the hand. Another 104 is around the base of the thumb. It can be secured by Velcro, button snaps, a belt, or any other methods of adjusting the size in a textile. To provide the user with a reliable grasp when using the system, the palmar side of the wearable 114 is made of a material with a high coefficient of friction, such as a rubber pad. The inside has a thin rubber lining around the edges of the glove to avoid slippage when worn on the hand.

The GUI 212 is installed in the connected device to guide users to perform the finger movements to initialize the machine learning algorithms, such as neural networks, to estimate the following finger movements. The GUI 212 functions as an interface to map the collected data to the control instructions of the connected device associated with the estimated finger movements. It provides users functions to config the map between the finger movement and the control instructions of the connected device, as well as checking signal quality, adjusting parameters of the machine learning algorithms, save and employ the collected data, etc., to help them use the system.

The proposed system provides a wearable way to recognize finger movements in real-time using sEMG signals and kinematic data from the back of the hand. Combined with machine learning algorithms, it provides a convenient and fast method to interface humans and machines.

One advantage of the system is that it can provide a faster way to transmit human intention compared to the mechanical movement of our hand by leveraging the phenomenon of electro-mechanical delay. In biology, the mechanical movement of the hand is powered by the muscles attached to the bone, which is activated by the signals from the nervous system. The proposed system collects sEMG signals, which contain the information of movement intension, by the sensor module 108, 110, 112. By applying machine learning algorithms on the short period of sEMG signals, the movement intention is captured from the proposed system. By limiting the duration of the sEMG signals required for each movement intension estimation, the delay from signal processing is constrained to be shorter than the electro-mechanical delay, which makes the proposed system capturing the movement intension earlier compared to from the mechanical movement of the hand. By circumventing the mechanical movement of the hand and directly communicating with the machines through the proposed system, the information transmission speed between the human brain and the machine is increased.

Another advantage of the system is that it can recognize the finger movement combining sEMG and kinematic data from the back of the hand and achieve the gesture control of the connected device. As there are more superficial muscles relating to the finger movement on the back of the hand than the forearm, it is easier for the machine learning algorithms to recognize the finger movement from the back of the hand compared to the forearm. The addition of the kinematic information would increase the estimation accuracy further.

In an example embodiment, the system is connected to the PC and could be used to increase the reaction time of the mouse click in gaming. The connection between the system and PC could be wired, such as USB cable, or wireless, such as Bluetooth. The PC needs to connect the mouse as well. The GUI 212 is installed on the PC. The user is asked to put the glove on the hand clicking the mouse. The user can adjust the strap to make the electrodes comfortably and firmly attached on the back of the hand. The noise level of the signals can be checked in the GUI 212. The user can relax himself/herself or slightly move or press the sensors to lower the noise.

If it is the first time for the user to use the system, it is required to collect some data for system calibration using GUI 212. In the data collection, each time GUI 212 would generate one point randomly displayed on the screen. The user needs to move the cursor to the point and make the required click, such as left, right, or thumb. The click type is also randomly generated. After around five to ten clicks for each type, the data collection is completed, and the collected data is processed for system calibration by the machine learning algorithm. The user could also select the stored data in the GUI 212 for model calibration.

During gameplay, if the user intends to make a click and the intension is captured by the proposed system, the GUI 212 will generate a click command to the PC. As the electro-mechanical delay, the timing of this “neural” click is ahead of the mechanical click from the mouse. As such, the PC will first accept the “neural” click. Meanwhile, the GUI 212 will intercept and disable the following mechanical click command from the mouse. As such, the mechanical click is replaced by the “neural” click. In another embodiment, the GUI does not disable the click from the mouse. The user intention, click, is transmitted to the PC by the proposed system instead of the mouse. As the timing advantage of neural information compared to the mechanical movement (the electro-mechanical delay), the game command initiated by the click, such as shooting (usually initiated by left mouse click), could be executed earlier. It is estimated to provide up to 60 milliseconds advantage to the users compared to mouse clicking. In shooting games, as the first player to initiate the shooting action is usually the player who will win a one-on-one engagement with another player, the “neural” click increase the chance of the user to win the game.

During gameplay, the user can configure the system to enable or disable the detection of each type of click, such as left, right or thumb. Shortcuts are provided to help users quickly achieve this. As the detection of each click type is ran in parallel, the system can simultaneously detect multiple types of clicks.

Another embodiment is to connect the system with the console, such as the Sony PlayStation Series, Microsoft Xbox Series. If it is the first time, the GUI 212 installed in the console would instruct the users to press buttons of the joystick using thumb, index, and middle finger of the hand with the glove on for data collection. During the data collection, each finger could press only one type of button. The model will be calibrated with the newly collected data or stored data in the GUI 212. After the model calibration, the system can detect the press button intension of the thumb, index, and middle finger. Once the intension is detected, for example, the intension of using the index finger to press Button A, the GUI 212 would generate a command to tell the console that Button A has been pressed and initiate the associated command in the game. Meanwhile, the GUI 212 would intercept and disable the following command from the joystick of pressing Button A. As such, the action associated with pressing Button A is initiated by the proposed system instead of the joystick. In another embodiment, the system does not disable the input from the controller. The user would have up to 60 milliseconds advantage to initiate the command with the “neural” press compared to the mechanical press. The user can config the disabling or enabling the function of detecting each finger press.

In the above two examples, the detection of the finger movement is to replace the mechanical movement to initiate the associated command of the connected device fast. Meanwhile, the command generated from the mechanical movement is disabled by the GUI 212 to avoid that it is executed twice. These two examples mainly take advantage of the electro-mechanical delay, circumventing the mechanical movement and increasing the information transmission speed from human to the machine.

Other embodiments are to use the proposed system to achieve remote control using hand and wrist movement. One example is to control the slide show. The proposed system is connected to the PC wirelessly, such as Bluetooth. The user needs to first wear the glove, adjust the straps to make it comfortable, and check the noise level of the signal in GUI 212. The user can relax himself/herself or slightly move or press the sensors to lower the noise.

If it's the first time of using the system, the user needs to collect data of different finger movements to calibrate the model of the algorithm. They can customize the mapping between the finger movement and the slide show control. For example, hand close is associated to open the slide show; wrist flexion is associated to go to the previous slide; wrist extension is associated to go to the next slide. After setting the mapping, the user only needs to collect the data of the movements registered in the mapping. The GUI 212 will guide the user to start and stop the performing of the movements. The user needs to perform the movement using the hand with the glove on. The collected data is used to calibrate the model of the algorithm. The user can also choose the stored data for model calibration.

In the slide show, if the user intends to open the slide, he/she can perform the hand close. If the movement, hand close, is detected by the proposed system, the GUI 212 will generate a command to the connected PC to open the slide. The user can also perform other movements registered in the mapping of the GUI 212 to control the slide show. As the glove and PC are wirelessly connected, the user can control the slide show remotely. The user can enable or disable the control in the GUI 212.

Another example is to control the smartphone. The glove is connected with the smartphone wirelessly. The GUI 212 is installed on the smartphone. Suppose the user would like to control the song playing. After wearing the glove, and checking the noise level, the user needs to set the mapping in the GUI 212, i.e. the relation between the finger movement and the play control. For example, hand close is associated to play the song; hand open is associated to stop playing; index finger flexion associated to play the next song. If it's the first time of using the system, the user needs to collect the data of the movement, hand close, hand open, and index finger flexion first registered in the mapping. The data is for model calibration of the algorithm. The user can also use the stored data in the GUI 212 for model calibration. After that, the user can control the song play by performing hand open, hand close, and index finger flexion, instead of pressing the touchscreen of the smartphone.

In the above two examples, the control command of the connected device is associated with the movements of the user by GUI 212. Once the user performs the registered movement, it will be detected by the proposed system through analyzing the collected data from sensor module 108, 110, 112 using machine learning algorithms. Then the system will ask the GUI 212 to send the command associated with the detected movement to the connected device to achieve the control. As the connection between the glove and the device is wireless, the control is remote.

In another embodiment, the user does not need to interface or use a GUI to be trained on the user data. Instead, training is initiated as soon as the user puts on the glove. The system will train either in the computer, or inside the controller of the device, and will begin sending commands based on the users muscle movements.

In another embodiment, the system can be trained with user data from a high volume of users, to create a general model that is able to work for any user without training. This version therefore would require no training at all for the individual user. They could put the glove on, and begin using it immediately.

The PC may be a general-purpose computer or a specialized computer, such as a gaming system. The GUI and other software described herein may be implemented in hardware or software, or a combination of both. These embodiments may be implemented in computer programs executing on programmable computers, each computer including at least one processor, a data storage system (including volatile memory or non-volatile memory or other data storage elements or a combination thereof), and at least one communication interface. Program code is applied to input data to perform the functions described herein and to generate output information. The output information is applied to one or more output devices, in known fashion.

Each program may be implemented in a high-level procedural or object oriented programming or scripting language, or both, to communicate with a computer system. However, alternatively the programs may be implemented in assembly or machine language, if desired. The language may be a compiled or interpreted language. Each such computer program may be stored on a storage media or a device (e.g., ROM, magnetic disk, optical disc), readable by a general or special purpose programmable computer, for configuring and operating the computer when the storage media or device is read by the computer to perform the procedures described herein. Embodiments of the system may also be considered to be implemented as a non-transitory computer-readable storage medium, configured with a computer program, where the storage medium so configured causes a computer to operate in a specific and predefined manner to perform the functions described herein.

Furthermore, the systems and methods of the described embodiments are capable of being distributed in a computer program product including a physical, non-transitory computer readable medium that bears computer usable instructions for one or more processors. The medium may be provided in various forms, including one or more diskettes, compact disks, tapes, chips, magnetic and electronic storage media, and the like. Non-transitory computer-readable media comprise all computer-readable media, with the exception being a transitory, propagating signal. The term non-transitory is not intended to exclude computer readable media such as a volatile memory or RAM, where the data stored thereon is only temporarily stored. The computer usable instructions may also be in various forms, including compiled and non-compiled code.

The scope of the claims should not be limited by the preferred embodiments set forth in description of the preferred embodiments or in the examples but should be given the broadest interpretation consistent with the description as a whole.

What is claimed is:

1. A user-wearable system for communicating with a computer system comprising
a hand wearable device;
a sensor array on the hand wearable device and configured to capture myoelectric signals and kinematic data from the user's hand; the sensor array positioned on the hand wearable device such that it is on a dorsal side of the user's hand when worn;
a means for converting the signals collected by the sensor array into computer-readable signals corresponding to a computer instruction executed by the computer system.

2. The system according to claim 1, wherein said means for converting comprises a control module on the hand wearable device.

3. The system according to claim 2, wherein said control module is configured to manage the data acquisition of each sensor on the sensor array and encode data received from each sensor.

4. The system according to claim 1, further comprising a transmission module for sending data to the computer system.

5. The system according to claim 4, where the transmission module communicates with the computer system by one of a wired, such as USB, or a wireless, such as BLE, WIFI, communication protocol.

6. The system according to claim 1, wherein said the means for converting further comprises machine learning algorithms, such as artificial neural networks (ANN), executed by the computer processor to decode data received from each sensor into the movements of the hand wearable.

7. The system according to claim 6, where the machine learning algorithm is calibrated in response to an initial capture of data by executing a calibration tool which provides the user with target movements of the hand wearable to correspond with visual cues on a graphical user interface, or the data stored in the graphical user interface.

8. The system according to claim 1, wherein said the means for converting further comprises a map between the movements of the hand wearable and the computer instructions which is configured in the graphical user interface by the user.

9. The system according to claim 8, where the graphical user interface translates the estimated movements from the machine learning algorithm to the computer-readable signals which corresponds with computer instructions based on the configured mapping.

10. The system according to claim 8, the computer instructions include mouse click and/or key/button press equivalent functions.

11. The system according to claim 1, wherein sensors in the sensor array captures myoelectric signals from the back of the hand, and the contacts for sensing is made of hard metals, such as silver or gold, or soft conductive materials, such as conductive silicone.

12. The system according to claim 1, wherein sensors further comprising a second sensor array for capturing kinematic data.

13. The system according to claim 12, wherein the captured myoelectric signal and the kinematic data are converted to digital signals with a sampling rate above 1000 Hz.

14. The system according to claim 1, wherein the hand wearable device consists of a textile with a first loop for an index finger and a second loop for a thumb.

15. The system according to claim 1, wherein sensors are positioned with respect to the finger loops.

16. The system according to claim 14, wherein the hand wearable device further includes a first adjustment strap at a side of the user's hand and a second adjustment strap around the base of the user's thumb.

17. The system according to claim 1, wherein said computer-readable signals transmit representations of human movements to the computer system which correspond with computer instructions.

18. The system according to claim 7 wherein said calibration tool stores models of different users and/or different uses of the system.
